(19) United States
(12) Patent Application Publication
(10) Pub. No.: US 2010/0076483 A1
(43) Pub. Date: Mar. 25, 2010

(54) MEDICAL MANIPULATOR

(75) Inventor: Shuji IMUTA, Fujinomiya-shi (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: TERUMO KABUSHIKI KAISHA, Shibuya-ku (JP)

(21) Appl. No.: 12/549,770

(22) Filed: Aug. 28, 2009

(30) Foreign Application Priority Data
Sep. 24, 2008 (JP) 2008-244035

(51) Int. Cl. A61B 17/28

(52) U.S. Cl. 606/205

(57) ABSTRACT

A medical manipulator includes an operating unit and a working unit. The operating unit has an actuator block with motors housed therein. The working unit is detachably mounted on the actuator block by a coupler. The working unit comprises a joint shaft that extends from the coupler, and a distal-end working unit mounted on a distal end of the joint shaft. The distal-end working unit has three axes, including a yaw axis, a roll axis about which the distal-end working unit is rotatable and which is positioned closer to a distal end of the distal-end working unit than the yaw axis, and a gripper axis about which a gripper is openable and closable and which is positioned on the distal end. The distal-end working unit can be operated about the roll axis by a motor, which is positioned closer to the distal end than the yaw axis.

### CROSS-REFERENCE TO RELATED APPLICATION

This application is based upon and claims the benefit of priority from Patent Application No. 2008-244035 filed on Sep. 24, 2008, in the Japan Patent Office, of which the contents are incorporated herein by reference.

### BACKGROUND OF THE INVENTION

1. Field of the Invention

The present invention relates to a medical manipulator having an actuator which incorporates proximal-end motors therein, and a working unit detachably coupled to the actuator by a coupler. More particularly, the present invention concerns a medical manipulator having a distal-end working unit on the distal end of a joint shaft which extends from the coupler.

2. Description of the Related Art

According to endoscopic surgery (also called laparoscopic surgery), it is customary to form a plurality of incisions in the body surface of the patient, insert trocars (tubular instruments) into the respective incisions as instrument passage ports, and to introduce distal ends of forceps having shafts through the respective trocars into the body cavity to perform a surgical operation on an affected part of the body. Working units such as a gripper for gripping living tissue, scissors, a blade of an electrosurgical knife, etc., are mounted on the distal ends of the forceps.

An endoscopic surgical operation performed with forceps requires the surgeon to be trained in advance because the working space inside the body cavity is small and the forceps need to be operated using the trocars as fulcrums. Since forceps that have been used heretofore have no joints in the working unit on the distal end thereof, the forceps have a small degree of freedom and the working unit can be operated only on an extension of the shaft. Therefore, cases that can be handled under usual training practices for endoscopic surgery are limited within a certain range, and a surgeon needs to be trained and highly skilled in order to be able to perform endoscopic surgery on various other cases not within the limited range.

Attempts have heretofore been made to improve conventional forceps, and develop a forceps having a plurality of joints in a working unit thereof (see, for example, Japanese Laid-Open Patent Publication No. 2004-105451 and Japanese Laid-Open Patent Publication No. 2008-036793). The manipulators disclosed in Japanese Laid-Open Patent Publication No. 2004-105451 and Japanese Laid-Open Patent Publication No. 2008-036793 comprise an operating unit which is manually operable, and a working unit replaceably mounted on the operating unit. The disclosed manipulators are free of the limitations and difficulties of conventional forceps, can be operated to perform easy surgical techniques, and can be applied to a wide variety of surgical cases. Such manipulators can be used to perform various surgical techniques by replacing the working unit with working units of different types.

The manipulators disclosed in Japanese Laid-Open Patent Publication No. 2004-105451 and Japanese Laid-Open Patent Publication No. 2008-036793 include a wire trained around and operatively extending between a proximal-end pulley coaxial with a motor disposed in the operating unit, and a distal-end pulley disposed in the distal-end working unit, for transmitting rotation of the proximal-end pulley to the distal-end pulley through the wire.

The distal-end working unit of the disclosed manipulators incorporates a three-axis mechanism, for example, having a yaw axis for swinging a working unit, a roll axis for rotating the working unit, and a gripper axis for opening and closing a gripper of the working unit. The three-axis mechanism makes it possible for the gripper to move in various directions.

There has been proposed a medical robot system for actuating such a manipulator with a robot arm (see, for example, U.S. Pat. No. 6,331,181).

In the manipulators disclosed in Japanese Laid-Open Patent Publication No. 2004-105451 and Japanese Laid-Open Patent Publication No. 2008-036793, the wire is trained around and operatively extends between the proximal-end pulley and the distal-end pulley. For accurately transmitting rotation of the motor to the distal-end working unit, the wire should not slip on the pulleys. To prevent the wire from slipping on the pulleys, the wire is welded or otherwise fixed at one point thereof to each of the pulleys, and is wound 1.5 or 2.5 turns around each of the pulleys in order to allow the pulleys to rotate through a suitable angular range.

As described above, the distal-end working unit of the disclosed manipulators incorporates a three-axis mechanism having a yaw axis, a roll axis, and a gripper axis. The yaw axis allows the working unit to swing thereabout through about ±90°, and the gripper axis allows the gripper to be opened and closed within an angular range from 0° to 90°. On the other hand, the roll axis should preferably allow the working unit to rotate endlessly within an infinite angular range. However, since the wire is welded or otherwise fixed at one point to each of the pulleys, the angle through which the pulleys are rotatable is limited by the number of turns of the wire around the pulleys. In particular, the distal-end pulley is disposed in a small space and hence has a thin shape, thereby limiting the number of turns of the wire to about 2.5 or 3.5. As a result, in reality, the roll axis is limited in rotation to ±180°.

Furthermore, the wire needs to be considerably small in diameter, since it is disposed in a joint shaft which interconnects the operating unit and the working unit. Under a torque applied from the motor, the thin wire tends to become elongated, and exhibits low power transmission efficiency, poor response, and has a short service life.

### SUMMARY OF THE INVENTION

It is an object of the present invention to provide a medical manipulator which makes it possible to allow a distal-end working unit to rotate endlessly within an infinite angular range about a roll axis.

A medical manipulator according to the present invention includes an actuator having a plurality of proximal-end motors, and a working unit detachably mounted on the actuator by a coupler. The working unit comprises a hollow joint shaft extending from the coupler, a distal-end working unit mounted on a distal end of the hollow joint shaft and having at least three axes including a roll axis about which the distal-end working unit is rotatable, and a distal-end motor mounted in the distal-end working unit, wherein the distal-end working unit is operated about the roll axis by the distal-end motor, and is operated about the at least three axes other than the roll axis by the proximal-end motors.

The proximal-end motors are positioned closer to a proximal end of the medical manipulator than the joint shaft, and the distal-end motor is disposed in the distal-end working unit. The distal-end working unit can be operated about the roll axis by the distal-end motor, and can be operated about axes other than the roll axis by the proximal-end motors. Since the distal-end motor is not limited to any rotational range, it can rotate the distal-end working unit endlessly within an infinite angular range about the roll axis.

The at least three axes may include a yaw axis, which is positioned closer to a proximal end of the distal-end working unit than the roll axis. The distal-end motor may be positioned closer to a distal end of the distal-end working unit than the yaw axis. With the distal-end motor being positioned closer to the distal end of the distal-end working unit than the yaw axis, the distal-end motor can appropriately rotate the distal-end working unit about the roll axis, even when the distal-end working unit is turned about the yaw axis, because the motor itself also is turned about the yaw axis.

The distal-end working unit may comprise a tubular body coaxial with the roll axis and angularly movable about the yaw axis, wherein the distal-end motor is disposed in the tubular body. The distal-end motor is stably held by the tubular body and can be angularly moved about the yaw axis.

The distal-end motor may have an output shaft coaxial with the roll axis. Accordingly, the distal-end working unit can be rotated about the roll axis by means of a simple mechanism.

The distal-end working unit may comprise a power transmitting tubular body coaxially disposed around the distal-end motor and having face gears disposed respectively on axial ends thereof, and a gripper openable and closable about a gripper axis positioned closer to a distal end of the distal-end working unit than the output shaft. The gripper may be opened and closed about the gripper axis by rotation of the power transmitting tubular body. The gripper, which is positioned closer to the distal end of the distal-end working unit than the output shaft, can thus be actuated appropriately.

The working unit may comprise a proximal-end rotor rotatable by the proximal-end motor, a distal-end rotor disposed in the distal-end working unit for operating the distal-end working unit about one of the at least three axes other than the roll axis, and a flexible member trained around the proximal-end rotor and the distal-end rotor for transmitting power from the proximal-end rotor to the distal-end rotor. The flexible member makes it possible to provide a simple, light, and inexpensive power transmitting mechanism within the working unit.

The medical manipulator may further comprise a hollow cylindrical member disposed in the joint shaft and rotatable by one of the distal-end motors, for operating the distal-end working unit about one of the at least three axes other than the roll axis. The hollow cylindrical member is less liable to suffer strains, and hence can transmit power highly efficiently.

The above and other objects, features, and advantages of the present invention will become more apparent from the following description when taken in conjunction with the accompanying drawings in which preferred embodiments of the present invention are shown by way of illustrative example.

### BRIEF DESCRIPTION OF THE DRAWINGS

FIG. 1 is a perspective view of a medical manipulator according to an embodiment of the present invention;

FIG. 2 is a side elevational view of the medical manipulator with a working unit and an operating unit being separate from each other;

FIG. 3 is a perspective view of the operating unit;

FIG. 4 is a perspective view, partially cut away, of a connector of the medical manipulator;

FIG. 5 is a perspective view of a distal-end working unit of the medical manipulator;

FIG. 6 is an exploded perspective view of the distal-end working unit;

FIG. 7 is a sectional side elevational view of the distal-end working unit;

FIG. 8 is a sectional plan view of the distal-end working unit;

FIG. 9 is a sectional side elevational view of a connector of the medical manipulator;

FIG. 10 is a perspective view of a proximal end portion of a power transmitting mechanism of the medical manipulator; and

FIG. 11 is a schematic perspective view of a medical robot system with the manipulator connected to the distal end of a robot arm.

### DESCRIPTION OF THE PREFERRED EMBODIMENTS

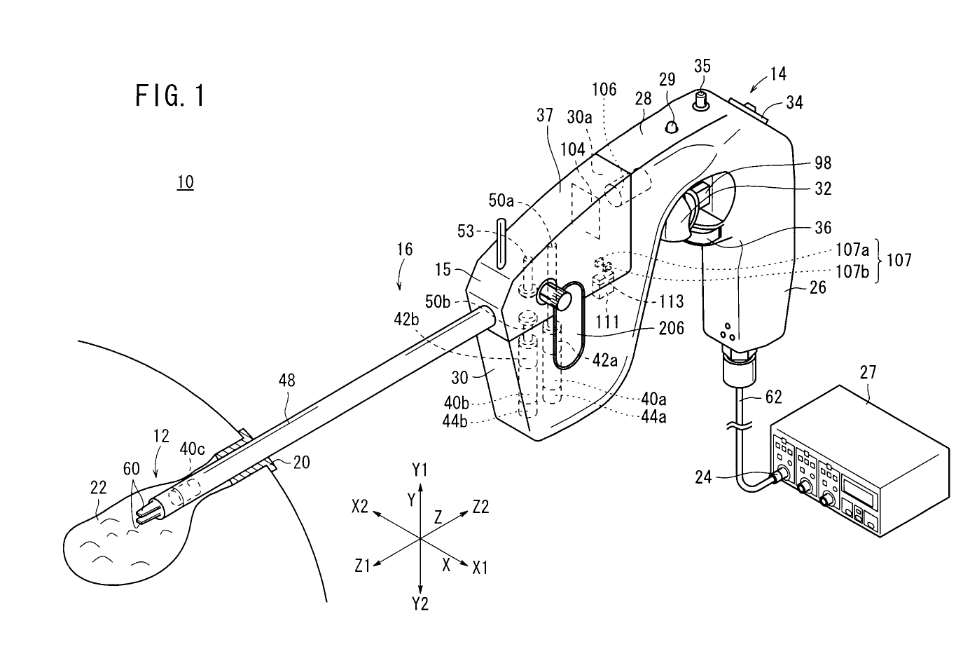

A medical manipulator according to an embodiment of the present invention will be described below with reference to FIGS. 1 through 11.

As shown in FIGS. 1, 2, and 3, the medical manipulator 10 has a distal-end working unit 12 for gripping a portion of a living tissue, a curved needle, or the like for performing a certain surgical treatment. The distal-end working unit 12 usually is referred to as a gripping forceps, a needle driver (needle holder), or the like.

The manipulator 10 comprises an operating unit 14 on a proximal end portion, which is held and operated by a human hand, and a working unit 16 detachably mounted on the operating unit 14. The operating unit 14 is electrically detachably connected to a controller 27 by a connector 24, thereby making up a manipulator system.

The manipulator 10 basically includes the operating unit 14 and the working unit 16. The controller 27 for electrically controlling the manipulator 10 is connected by the connector 24 to a cable 62, which extends from the lower end of a grip handle 26 of the operating unit 14. Some or all of the functions of the controller 27 may be incorporated into the operating unit 14.

It shall be assumed in the following description that transverse directions in FIG. 1 are referred to as X directions, vertical directions as Y directions, and longitudinal directions of a hollow joint shaft 48 as Z directions. Among the X directions, the rightward direction as viewed from the distal end is referred to as an X1 direction, and the leftward direction as an X2 direction. Among the Y directions, the upward direction is referred to as a Y1 direction, and the downward direction as a Y2 direction. Among the Z directions, the forward direction is referred to as a Z1 direction, and the rearward direction as a Z2 direction. Unless otherwise noted, these directions represent directions of the manipulator 10 when it is placed in a reference attitude (neutral attitude). The above directional definitions are for illustrative purposes only. The manipulator 10 can be used in any orientation, e.g., it may be used upside down.

The working unit 16 comprises a distal-end working unit 12 for performing a working operation, a coupler 15 connected to an actuator block (actuator) 30 of the operating unit 14, and an elongate hollow joint shaft 48 coupling the distal-end working unit 12 and the coupler 15 to each other. When a predetermined action is performed on the actuator block 30, the working unit 16 can be separated from the operating unit 14, so that the working unit 16 can be cleaned, sterilized, and serviced for maintenance.

The distal-end working unit 12 and the joint shaft 48, which are small in diameter, can be inserted into a body cavity 22 through a trocar 20 in the form of a hollow cylinder mounted in an abdominal region or the like of the patient. The distal-end working unit 12 is actuated by the operating unit 14 to perform various surgical techniques to remove, grip, suture, or ligate an affected part of the patient's body in the body cavity 22.

The operating unit 14 will be described in detail below.

The operating unit 14 includes a grip handle 26 gripped by a human hand, a bridge 28 extending from an upper portion of the grip handle 26, and an actuator block 30 connected to a distal end of the bridge 28.

The coupler 15 has two engaging teeth 200 disposed respectively on opposite side surfaces thereof, and three fitting holes 202a, 202b, 202b defined therein, which are open at upper and lower surfaces of a rotor housing 250 described later. The three fitting holes 202a, 202b, 202c are disposed near ends of the coupler 15 in the Z1 and Z2 directions, and extend in the Y directions.

The actuator block 30 houses therein two motors (proximal-end motors) 40a, 40b, which extend parallel to each other and are arrayed at spaced intervals along the Z directions. The motors 40a, 40b, which are operatively associated with respective mechanisms disposed in the distal-end working unit 12, are energized under the control of the controller 27 based on actions made by the operator on the operating unit 14. The motors 40a, 40b are small in size and diameter, and the actuator block 30, which houses the motors 40a, 40b therein, has a flat compact shape.

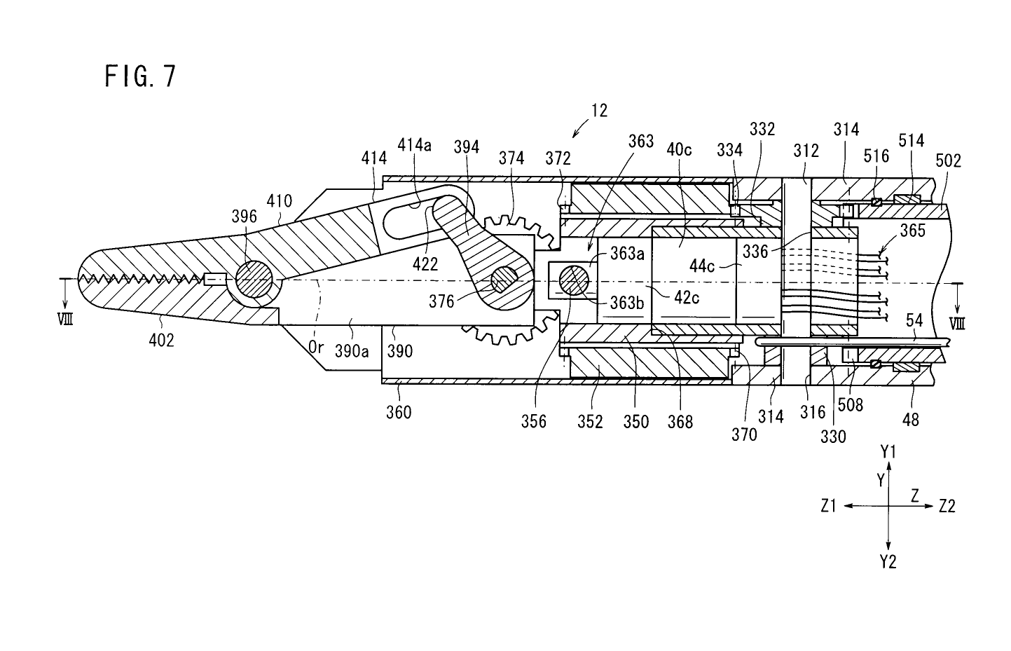

As described in detail later with reference to FIG. 7, the distal-end working unit 12 also houses therein a motor (distal-end motor) 40c. The motors 40a, 40b, 40c operate independently or cooperatively to actuate three-axis mechanisms disposed in the distal-end working unit 12. Each of the motors 40a, 40b, 40c comprises a DC motor, for example.

The motors 40a, 40b, 40c are combined with respective speed reducers 42a, 42b, 42c each in the form of a planetary gear assembly, for example, having a speed reduction ration ranging from 1:100 to 1:300.

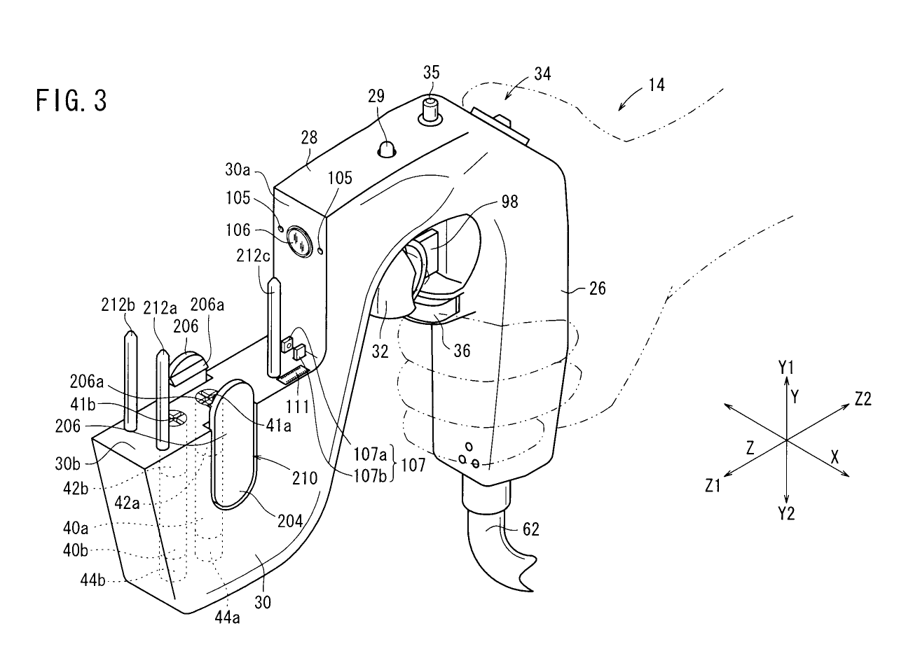

The actuator block 30 is disposed downwardly of the end of the operating unit 14 in the Z1 direction. The actuator block 30 implies a body on which the working unit 16 is mounted, and is not limited to a structure that stores the motors 40a, 40b therein, but covers a joint surface 30a (see FIG. 3) connected to the bridge 28.

The motors 40a, 40b, 40c also are combined with respective rotary encoders 44a, 44b, 44c for detecting angular displacements of the respective drive shafts of the motors 40a, 40b, 40c. The rotary encoders 44a, 44b, 44c supply detected angle signals to the controller 27.

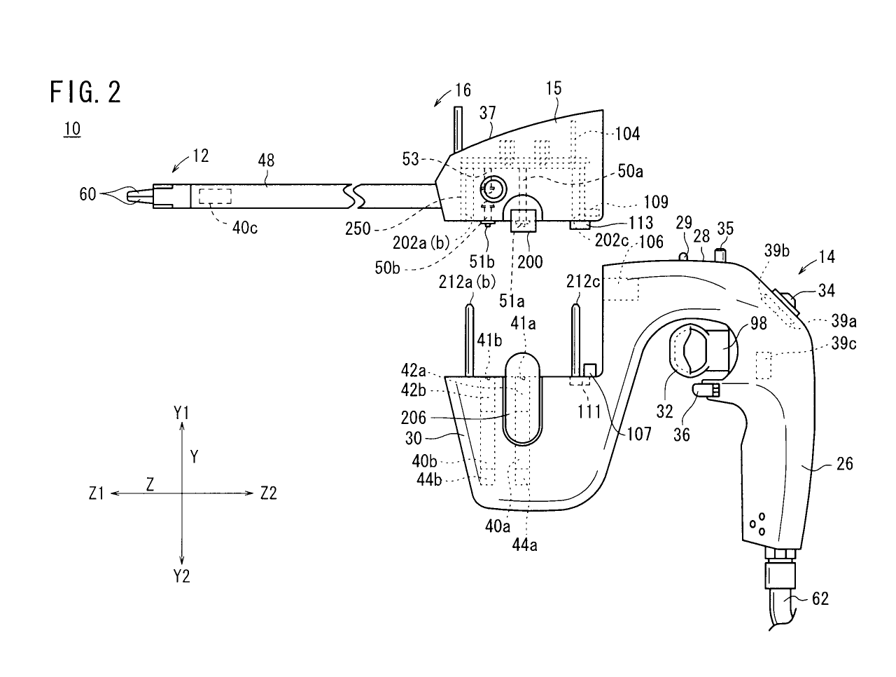

As shown in FIGS. 2 and 3, the grip handle 26 extends in the Y2 direction from the end of the bridge 28, and has a length suitable for being gripped by a human hand. The grip handle 26 has an input means for entering signals for operating the distal-end working unit 12. The input means includes a trigger lever 32 and a switch 36 disposed closely to the grip handle 26 and projecting away from the grip handle 26 in the Z1 direction, and a composite input unit 34 and an operation switch 35 facing away from the grip handle 26 in the Y1 direction.

An LED 29 is mounted on the upper surface of the bridge 28 at a location that can easily be viewed by the operator of the manipulator 10. The LED 29 is spaced from the operation switch 35 in the Z1 direction. The LED 29 serves as an indicator for indicating a controlled state of the manipulator 10. The LED 29 has a size large enough to be easily visually recognizable by the operator, and yet is sufficiently small and light not to interfere with operation of the manipulator 10.

The cable 62 connected to the controller 27 has an end connected to the lower end of the grip handle 26. The grip handle 26 and the cable 62 may be connected to each other by a connector.

The operation switch 35 serves to selectively enable or disable the manipulator 10. The LED 29 is located in a visually recognizable position substantially centrally on the upper surface of the bridge 28, in a juxtaposed relation to the operation switch 35. For example, the LED 29 is turned on in synchronism with the operation switch 35 when the operation switch 35 is turned on. Therefore, when the operator turns on or off the operation switch 35, the operator can reliably recognize and confirm that the operation switch 35 has been turned on or off by visually checking the LED 29.

The controller 27 reads the state of the operation switch 35. When the operation switch 35 is turned on, the controller 27 sets the manipulator 10 in an operation mode. When the operation switch 35 is turned off, the controller 27 sets the manipulator 10 in an automatic origin return mode, and returns the motors 40a, 40b, 40c to their origins. After the motors 40a, 40b, 40c have been returned to their origins, the controller 27 sets the manipulator 10 in a stop mode. In the operation mode, the controller 27 enables operation commands entered from the operating unit 14 to energize the motors 40a, 40b, 40c. In the stop mode, the controller 27 stops the motors 40a, 40b, 40c regardless of whether or not operation commands are entered from the operating unit 14. The controller 27 distinguishes these modes and switches between different energized states of the LED 29 based on the distinguished modes.

Specifically, when the manipulator 10 is in the operation mode, the controller 27 energizes the LED 29 to emit green light. When the manipulator 10 is in the stop mode, the controller 27 energizes the LED 29 to emit red light. When the manipulator 10 is in the automatic origin return mode, upon switching from the operation mode to the stop mode, the controller 27 energizes the LED 29 to emit flickering red light.

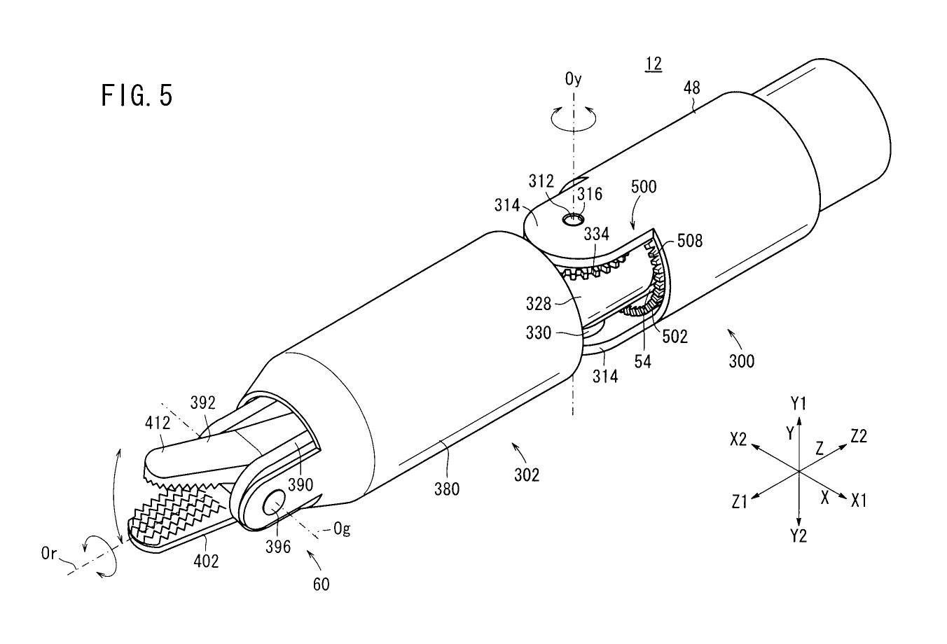

The composite input unit 34 serves as a composite input means for imparting rotational commands in rolling directions (shaft rotating directions) and yawing directions (left and right directions) to the distal-end working unit 12. The composite input unit 34 includes, for example, a first input means, which operates in shaft rotating directions for imparting rotational commands in rolling directions, and a second input means, which operates in left and right directions for imparting rotational commands in yawing directions. The trigger lever 32 serves as an input means for imparting opening and closing commands to a gripper 60 (see FIGS. 1 and 5) of the distal-end working unit 12.

The composite input unit 34 and the trigger lever 32 are combined with input sensors 39a, 39b, 39c (see FIG. 2) for detecting strokes of movement thereof. The input sensors 39a, 39b, 39c supply detected stroke signals to the controller 27.

The trigger lever 32 is disposed slightly below the bridge 28 and projects in the Z1 direction. The trigger lever 32 is disposed in a position where the trigger lever 32 can easily be operated by the index finger of a hand that is gripping the grip handle 26.

The trigger lever 32 is operatively connected to the grip handle 26 by an arm 98, and is movable toward and away from the grip handle 26. The arm 98 is connected to the input sensor 39c in the grip handle 26. The distance that the trigger lever 32 has moved is detected by the input sensor 39c, which supplies a signal representing the detected distance to the controller 27. The trigger lever 32 can be pulled toward the grip handle 26 in the Z2 direction by the finger, and can be pushed away from the grip handle 26 in the Z1 direction by the finger. When the trigger lever 32 is thus pulled or pushed, the controller 27 receives a signal from the input sensor 39c, and imparts opening and closing commands to the gripper 60.

The switch 36, which is spaced from the trigger lever 32 in the Y2 direction, comprises an alternate switch. When the switch 36 is operated, the gripper 60 remains in a certain state, e.g., in a closed state, which is brought about by the trigger lever 32.

A working unit detecting means 107, for detecting whether or not the coupler 15 is placed on the actuator block 30, is disposed on an upper surface 30b of the actuator block 30 at an end thereof in the Z2 direction. The working unit detecting means 107 comprises an LED 107a as a light emitter and a photodiode 107b as a light detector, which are positioned in confronting relation to each other in a structure of a photo interrupter. When a light shield 109 (see FIG. 2) in the rear end of the coupler 15 is inserted between the LED 107a and the photodiode 107b, the light shield 109 blocks light emitted from the LED 107a toward the photodiode 107b, thereby detecting that the coupler 15 is mounted on the actuator block 30. The LED 107a and the photodiode 107b confront each other in the X directions, and are disposed closely to each other.

A connector 111 connected to the controller 27 and oriented in the Y1 direction is disposed on the upper surface 30b.

The actuator block 30 has a pair of independent engaging fingers 210 for holding the coupler 15 of the working unit 16, and three alignment pins 212a, 212b, 212c for positioning and holding the coupler 15.

The two engaging fingers 210 are pivotally mounted in symmetrical positions on respective outer side surfaces thereof, which face in the X1 and X2 directions. The engaging fingers 210 comprise respective pusher surfaces 204 and respective levers 206 that extend in the Y1 direction from the pusher surfaces 204. The levers 206 project slightly from the upper surface of the actuator block 30 in the Y1 direction, and have respective wedges 206a on upper inner tapered surfaces thereof for engaging with the respective engaging teeth 200 on outer side surfaces of the coupler 15 when the coupler 15 is mounted on the actuator block 30. The engaging fingers 210 are normally biased by resilient members, not shown, to displace the levers 206 inwardly toward each other.

The alignment pins 212a, 212b, 212c are disposed in alignment with the respective fitting holes 202a, 202b, 202c. Among the three alignment pins 212a, 212b, 212c, two alignment pins 212a, 212b are disposed near the end of the upper surface of the actuator block 30 in the Z1 direction, whereas the alignment pin 212c is disposed near the other end of the upper surface of the actuator block 30 in the Z2 direction. The alignment pins 212a, 212b, 212c extend in the Y1 direction. Also, the alignment pins 212a, 212b, which are disposed near the end of the upper surface of the actuator block 30 in the Z1 direction, are spaced from each other in the X directions.

Since the actuator block 30 has the three alignment pins 212a, 212b, 212c, the coupler 15 is supported by the actuator block 30 at three positions corresponding to the alignment pins 212a, 212b, 212c, and the coupler 15 is simply and reliably positioned with respect to the actuator block 30. As the three alignment pins 212a, 212b, 212c are not positioned in a linear array, but are positioned in a triangular pattern, the alignment pins 212a, 212b, 212c can hold the coupler 15 stably against twisting forces applied in any direction. At least two of the alignment pins 212a, 212b, 212c may be effective to reliably position and hold the coupler 15 stably on the actuator block 30. If two such alignment pins are spaced from each other in the Z directions, then the alignment pins are effective to hold the coupler 15 more stably on the actuator block 30.

For removing the coupler 15 from the operating unit 14, the operator presses the pusher surfaces 204 of the engaging fingers 210 simultaneously toward each other in order to tilt the levers 206 against the resiliency of the resilient members and to bring the wedges 206a out of engagement with the engaging teeth 200. The coupler 15 can then be pulled and removed from the operating unit 14 upwardly in the Y1 direction. While the coupler 15 is disposed on the actuator block 30, since the three alignment pins 212a, 212b, 212c on the upper surface 30b of the actuator block 30 are fitted respectively into the fitting holes 202a, 202b, 202c in the coupler 15, the coupler 15 is stably held on the actuator block 30.

For connecting the coupler 15 to the operating unit 14, the operator aligns the alignment pins 212a, 212b, 212c respectively with the fitting holes 202a, 202b, 202c, and inserts the alignment pins 212a, 212b, 212c respectively into the fitting holes 202a, 202b, 202c by lowering the coupler 15 in the Y2 direction. The levers 206 of the engaging fingers 210 are displaced outwardly while sliding on the outer surfaces of the engaging teeth 200. Then, the levers 206 snap back under resiliency of the resilient members, thereby bringing the wedges 206a into engagement with the engaging teeth 200. The coupler 15 thus becomes completely mounted on the actuator block 30.

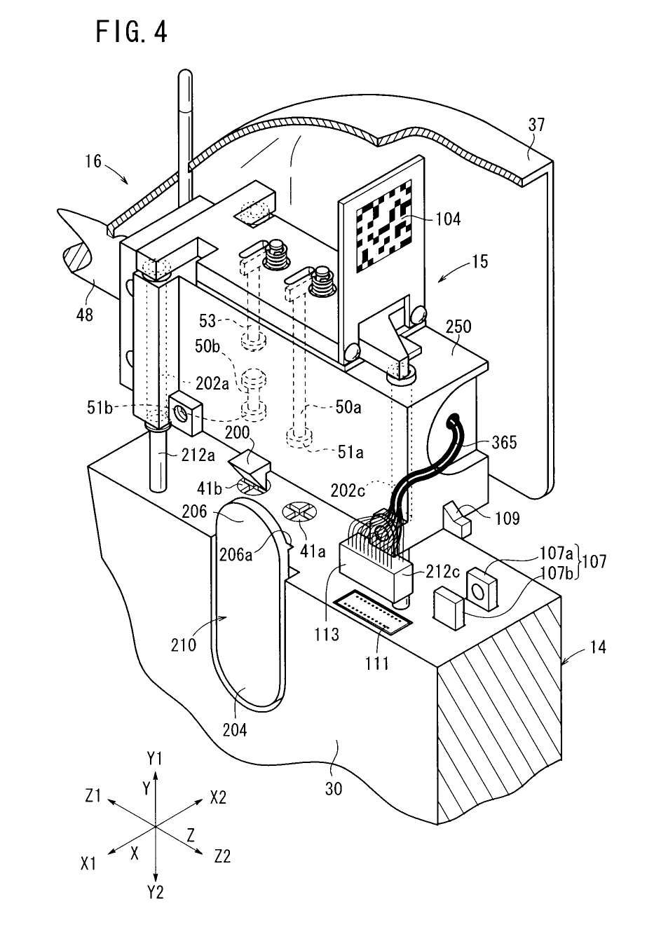

On the joint surface 30a of the operating unit 14, a camera 106 is mounted for reading a QR code of an ID card 104 (see FIG. 4) of the connected working unit 16 and supplying the read QR code to the controller 27, together with two LEDs 105 for illuminating the ID card 104 of the connected working unit 16. The camera 106 is disposed in a position facing the ID card 104, and the LEDs 105 are positioned one on each side of the camera 106. The camera 106 may be replaced with a bar-code reader or a bar-code scanner for reading an ID code of the ID card 104.

The operating unit 14, which houses several electric components therein, is more costly but has a longer service life than the working unit 16.

The working unit 16 will be described in detail below. After a surgical operation using the manipulator 10 is finished, the working unit 16 can be removed from the operating unit 14 and cleaned. The working unit 16 may also periodically be replaced with a new working unit 16 for achieving sufficient reliability. Since the working unit 16 does not include electronic devices, the working unit 16 is inexpensive. In addition, in view of the mechanical service life of the working unit 16, which is affected by the distal-end working unit 12 as it suffers undue burdens due to operations in the body cavity 22, and also in view of damage caused when the working unit 16 is cleaned with steam and heat, the working unit 16 can be replaced with a new working unit 16 at appropriate times. In view of the general service life of the working unit 16, the manufacturer of the working unit 16 sets a limit on the number of times that the working unit 16 can be used repeatedly. A managerial person, who works for the medical organization that uses the manipulator 10, counts the number of times that the working unit 16 has been used, and disposes of the working unit 16 according to a given procedure when the limit number is reached or exceeded.

As shown in FIGS. 1, 2, and 4, the coupler 15 of the working unit 16 is covered with a resin cover 37. The coupler 15 houses a proximal-end pulley (proximal-end rotor) 50a, which is supported rotatably therein and is connected to the motor 40a and can be rotated thereby, a drive shaft 50b, which is supported rotatably therein and is connected to the motor 40b and can be rotated thereby, and a driven shaft 53 disposed coaxially within the drive shaft 50b. The proximal-end pulley 50a, the drive shaft 50b, and the driven shaft 53 are housed in the rotor housing 250 of the coupler 15.

The proximal-end pulley 50a and the drive shaft 50b in the coupler 15 have respective criss-cross coupling teeth 51a, 51b on lower ends thereof in the Y2 direction, and the rotatable shafts of the motors 40a, 40b in the actuator block 30 have respective criss-cross coupling recesses 41a, 41b. The coupling teeth 51a, 51b can engage within the respective coupling recesses 41a, 41b. When the coupler 15 is mounted on the actuator block 30, the coupling teeth 51a, 51b engage within the respective coupling recesses 41a, 41b for transmitting rotation of the motors 40a, 40b to the proximal-end pulley 50a and the drive shaft 50b. The coupling teeth 51a, 51c and the coupling recesses 41a, 41b may have shapes other than criss-cross shapes.

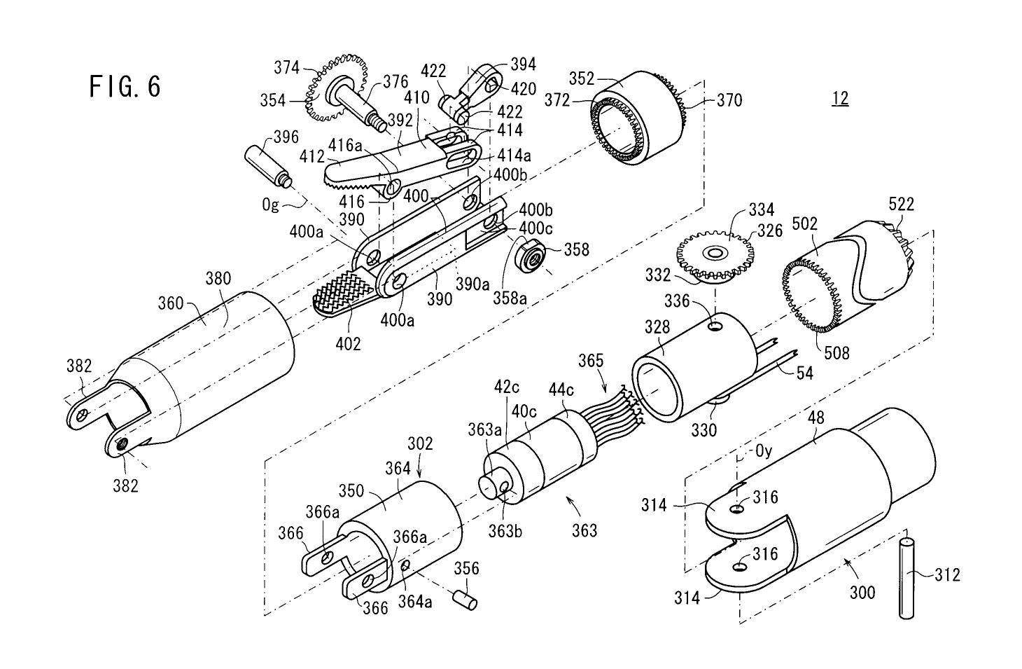

A wire (flexible member) 54 extends through the joint shaft 48 and a hollow cylindrical member 502 (see FIGS. 5 and 6), and is trained around the proximal-end pulley 50a and a distal-end pulley 330, to be described later, for transmitting power from the proximal-end pulley 50a to the distal-end pulley 330. The wire 54 is trained 1.5 turns around the proximal-end pulley 50a and the distal-end pulley 330, respectively, so as to allow the proximal-end pulley 50a and the distal-end pulley 330 to rotate through a sufficient angular range. The wire 54 is welded at certain points thereof to the proximal-end pulley 50a and the distal-end pulley 330 to prevent slippage. The wire 54 makes it possible to provide a simple, light, and inexpensive power transmitting mechanism in the working unit 16.

Rotation of the drive shaft 50b is transmitted to the distal-end working unit 12 by the hollow cylindrical member 502 (see FIGS. 5 and 6).

As shown in FIG. 4, the ID card 104, which carries an ID (identification) mark for identifying the individual working unit 16, is disposed on the coupler 15 proximate the rear end thereof.

The ID mark carried by the ID card 104 comprises a QR code in the form of a two-dimensional bar code for identifying the working unit 16. The QR code is peculiar to one working unit 16, and hence different QR codes are assigned to different working units 16, respectively. The QR code contains various pieces of information, including the type, specifications, serial number, production factory, production date, tradename, etc., of the working unit 16.

A connector 113, which is connected to a motor assembly 363 (see FIG. 6) and oriented in the Y2 direction, is mounted on a lower portion of a side surface of the rotor housing 250. The motor assembly 363 includes the motor 40c. When the coupler 15 is mounted on the actuator block 30, the connector 113 and the connector 111 are automatically interfitted, and electrically connected for transmitting signals from the motor assembly 363 to the controller 27 and for transmitting a drive signal from the controller 27 to the motor 40c. When the operator installs the coupler 15 on the actuator block 30 and removes the coupler 15 from the actuator block 30, the operator does not need to be concerned about the connectors 111, 113, and does not need to undertake actions to connect and disconnect the connectors 111, 113.

As shown in FIG. 5, the distal end of the joint shaft 48 has a pair of spaced tongues 314 that project toward the distal end of the working unit 16, and which are disposed one on each side of the central axis of the joint shaft 48 in facing relation thereto. The tongues 314 have respective shaft holes 316 defined therein in alignment with each other. The tongues 314 have respective arcuate distal end edges, and respective flat inner surfaces facing each other and lying in parallel with each other. The shaft holes 316 are spaced from each other across the central axis of the joint shaft 48.

The distal-end working unit 12 incorporates therein mechanisms having three degrees of freedom. Such mechanisms include a yaw-axis mechanism 300 having a first degree of freedom for angularly moving a distal end portion positioned ahead of a first rotational axis Oy, i.e., a yaw axis, extending along the Y directions, in yawing directions about the first rotational axis Oy, a roll-axis mechanism 302 having a second degree of freedom for angularly moving the distal end portion in rolling directions about a second rotational axis Or, i.e., a roll axis, extending along the Z directions, and a gripper opening and closing mechanism having a third degree of freedom for opening and closing the gripper 60 on the distal end about a third rotational axis Og, i.e., a gripper axis, extending along the X directions.

The yaw-axis mechanism 300, the roll-axis mechanism 302, and the gripper opening and closing mechanism, which are incorporated in the distal-end working unit 12, will be described in detail below with reference to FIGS. 5, 6, 7, and 8.

The yaw-axis mechanism 300 is disposed between the tongues 314. The yaw-axis mechanism 300 includes a shaft 312, which is inserted in the shaft holes 316. The shaft 312 is secured in the shaft holes 316 by press fitting, for example. The shaft 312 is coaxial with the first rotational axis Oy.

The yaw-axis mechanism 300 includes a gear assembly 326 rotatably supported on the shaft 312, a motor holding tube (tubular body) 328, and the distal-end pulley (distal-end rotor) 330, which are arranged successively in the Y2 direction. The motor holding tube 328 and the distal-end pulley 330 are combined integrally with each other.

The gear assembly 326 comprises a tubular body 332, and a gear 334 disposed concentrically on an upper surface of the tubular body 332. The gear 334 has a thin structure. The gear assembly 326 is rotated about the shaft 312 when rotation of the motor 40b is transmitted through the hollow cylindrical member 502 to the gear 334.

The motor holding tube 328 comprises a hollow tubular body, which holds the motor assembly 363 therein. The distal-end pulley 330 comprises a short cylindrical body on which a portion of the wire 54 is fixedly wound. The motor holding tube 328 extends in the Z directions. The distal-end pulley 330 is disposed near the rear end of the motor holding tube 328 and is oriented in the Y2 direction. The motor holding tube 328 and the distal-end pulley 330 have a hole 336 defined therein through which the shaft 312 is inserted.

The roll-axis mechanism 302, which also may be referred to as a drive mechanism, comprises a drive base 350, a gear ring (power transmitting tubular body) 352, a geared pin 354, a fastening nut 358, a cover 360, and the motor assembly 363. The fastening nut 358 has a pair of parallel surfaces 358a, which are engageable by a turning tool such as a wrench or the like.

The motor assembly 363 is made up of the motor 40c, the speed reducer 42c combined with the motor 40c for transmitting rotation of the motor 30c at a speed reduction ratio, and the rotary encoder 44c for measuring the angular displacement of the motor 40c. The motor assembly 363 is in the form of an integral cylindrical structural body, in which the rotary encoder 44c, the motor 40c, and the speed reducer 42c are arranged successively in the Z1 direction.

The motor assembly 363 includes an output shaft 363a projecting from an end thereof in the Z1 direction. The output shaft 363a has a hole 363b defined in a side surface thereof. A plurality of power lines connected to the motor 40c, and a plurality of signal lines connected to the rotary encoder 44c are bundled as a harness 365, which extends through the joint shaft 48 in the Z2 direction from the end of the motor assembly 363 to the connector 113 in the coupler 15.

The drive base 350 comprises a tubular body 364, which is rotatably disposed around the output shaft 363a of the motor assembly 363, and a pair of support arms 366 projecting in the Z1 direction from respective lateral opposite ends of the tubular body 364. The tubular body 364 has a hole 364a extending through a side wall thereof. The support arms 366, which serve to support the gripper 60, have respective holes 366a defined therein, which are arrayed in the X directions.

The tubular body 364 has a rear end portion fitted over and rotatably disposed on the motor holding tube 328. The tubular body 364 has a step 368 (see FIG. 7) defined in an inner wall surface thereof for determining the axial distance by which the rear end portion of the tubular body 364 is fitted over the motor holding tube 328. After the tubular body 364 is placed over the motor holding tube 328, the fastening pin 356 is press-fitted into the hole 364a in the tubular body 364 and into the hole 363b in the output shaft 363a, thereby connecting the tubular body 364 to the output shaft 363a. The drive base 350 is thus angularly movable in rolling directions by the motor 40c about the axis of the output shaft 363a, i.e., about the second rotational axis Or.

The gear ring 352 is in the form of a thin tubular body having a face gear 370 on a surface thereof facing in the Z2 direction, and a face gear 372 on a surface thereof facing in the Z1 direction. The gear ring 352 is fitted over the tubular body 364 of the drive base 350, and is slidably rotatable around the outer circumferential surface of the tubular body 364. The face gear 370 is held in mesh with the gear 334 for rotation about the second rotational axis Or upon rotation of the gear assembly 326.

The geared pin 354 has a gear 374 that meshes with the face gear 372, and a pin 376 extending in the X1 direction from the center of the gear 374. The pin 376 has an externally threaded distal end portion. The pin 376 extends through the holes 366a in the respective support arms 366, and the externally threaded distal end portion thereof projects from one of the support arms 366. The fastening nut 358 is threaded over the projecting externally threaded distal end of the pin 376. With the pin 376 thus assembled, the gear 374 is held in mesh with the face gear 372, and the pin 376 is rotatably supported on the support arms 366. The pin 376 has a D-shaped cross section for driving engagement with a portion of the gripper 60.

The cover 360 serves to protect the various components of the roll-axis mechanism 302 described above, and covers the gear ring 352 and the gear 374 against exposure in radial directions of the distal-end working unit 12. The cover 360 comprises a short sleeve 380 extending in the Z2 direction, and a pair of lobes 382 projecting in the Z1 direction from respective lateral opposite ends of the short sleeve 380. The lobes 382 are shaped as axial extensions of portions of the circumferential wall of the short sleeve 380, and have a radius of curvature identical to that of the circumferential wall of the short sleeve 380. The cover 360 is secured by a pin 396 to a portion of the gripper 60. The cover 360 is identical to or smaller in diameter than the joint shaft 48.

When the motor 40c is energized, the motor 40c rotates the drive base 350 and the gripper 60 connected to the drive base 350 about the second rotational axis Or. When the gear 334 is rotated, rotation of the gear 334 is transmitted through the face gear 370, the face gear 372, and the gear 374 to the pin 376, thereby rotating the geared pin 354.

The gripper 60 comprises a first end effector 390, a second end effector 392, a link 394, and the pin 396. The pin 396 is aligned with the third rotational axis Og.

The first end effector 390 comprises a pair of laterally spaced side walls 400 facing each other and having respective holes 400a defined in distal end portions thereof, and respective holes 400b defined in proximal end portions thereof, a first gripper 402 projecting in the Z1 direction from lower portions of the distal ends of the side walls 400, and a cover fixture 404 disposed on lower portions of the proximal ends of the side walls 400. The diameter of the holes 400a is set to be a diameter such that the pin 396 is suitably press-fitted therein, for example. The first gripper 402 is progressively narrower in the Z1 direction, and has an arcuate distal end portion. The first gripper 402 includes a number of small pyramidal teeth closely disposed fully over the surface thereof facing in the Y1 direction.

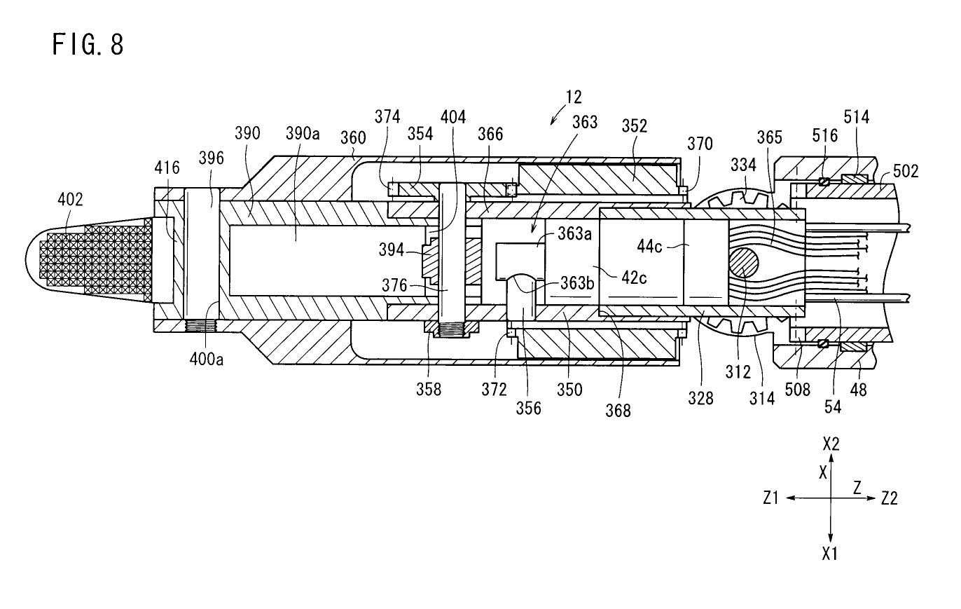

The distal ends of the side walls 400 are arcuate in shape. The side walls 400 have respective recesses 400c defined respectively in outer surfaces thereof for receiving respective support arms 366 therein. The first end effector 390 includes a hole 390a (see FIG. 8) defined between the first gripper 402 and the cover fixture 404 for preventing the first end effector 390 from interfering with the proximal end of the second end effector 392. The cover fixture 404 has a hole in which the pin 376 may be press-fitted, for example.

The second end effector 392 comprises a base 410, a second gripper 412 extending in the Z1 direction from a distal end of the base 410, a pair of lobes 414 extending in the Z2 direction from respective laterally opposite ends of the base 410, and a pin support tube 416 disposed on a lower surface of the distal end of the base 410. The pin support tube 416 has a hole 416a defined therein, which has an inside diameter large enough to receive the pin 396 when the pin 396 is inserted therein. When the pin 396 is inserted into the hole 416a and press-fitted, for example, in the holes 400a of the first end effector 390, the second end effector 392 is swingable about the third rotational axis Og. The second gripper 412 is identical in shape to the first gripper 402, and is oriented in a vertically inverted relationship with respect to the first gripper 402. When the second end effector 392 is angularly moved counterclockwise in FIG. 7 about the third rotational axis Og, the second gripper 412 is brought in close proximity to the first gripper 402, jointly gripping a curved needle or the like therebetween. The lobes 414 have respective oblong holes 414a defined therein.

The link 394 has a hole 420 defined in one end thereof, and a pair of engaging fingers 422 projecting laterally in opposite directions from the other end thereof. The engaging fingers 422 slidably engage within the respective oblong holes 414a in the lobes 414. The hole 420 has a D-shaped cross section for engagement with the pin 376, thereby providing a function to position the pin 376, and a function to stop the pin 376 from rotating within the hole 420. When the pin 376 is inserted in the holes 366a, 400b, 420, and the fastening nut 358 is threaded over the distal end of the pin 376, the link 394 is swingable about the pin 376.

The hollow cylindrical member 502 and peripheral mechanisms thereof for transmitting rotation of the motor 40b to the gear assembly 326 will be described below.

The hollow cylindrical member 502 has an outside diameter such that the hollow cylindrical member 502 is substantially held in contact with the inner surface of the joint shaft 48, and is rotatable about its own axis in the joint shaft 48. The hollow cylindrical member 502 is made of a metal such as an aluminum alloy, stainless steel, or the like, or a high-strength material such as carbon fiber resin or the like, and has a larger cross-sectional area, suffers less strain, and is higher in rigidity than thin filamentary members such as wires. Therefore, the hollow cylindrical member 502 has high power transmission efficiency, a high response, and a long service life.

The hollow cylindrical member 502 has a face gear 508 on the distal end thereof for meshing with the gear 334 of the gear assembly 326 at a portion facing in the Z2 direction. The hollow cylindrical member 502 is supported in the joint shaft 48 by a slide bearing 514.

An O-ring (seal member) 516 is interposed between the joint shaft 48 and the hollow cylindrical member 502.

The O-ring 516 is positioned closer to the distal end of the hollow cylindrical member 502 than the slide bearing 514, in order to prevent foreign matter and solidified body fluids from becoming caught by the sliding surfaces of the slide bearing 514.

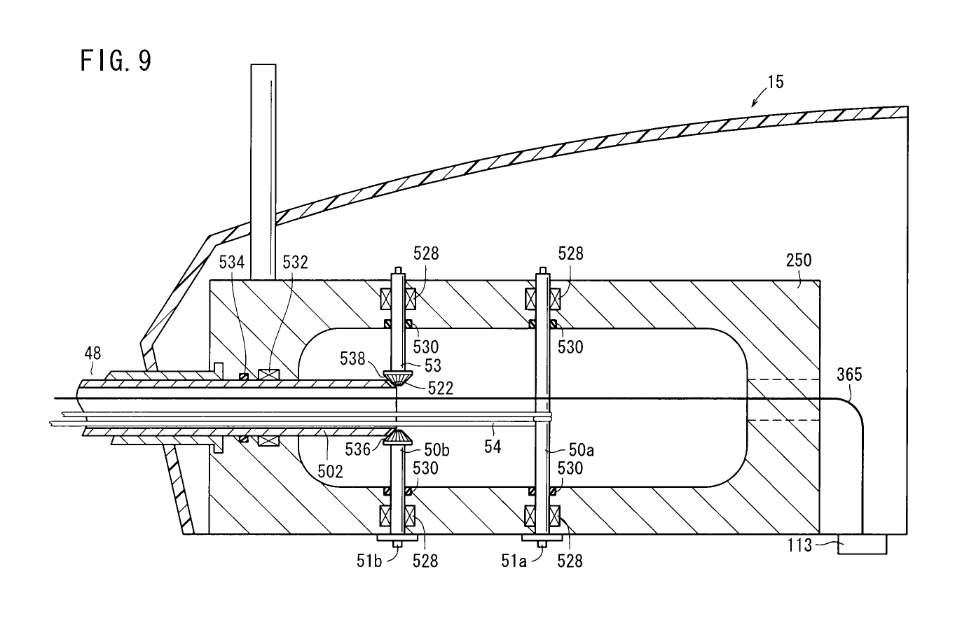

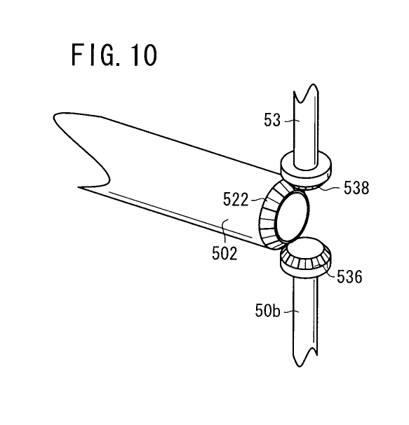

As shown in FIGS. 9 and 10, the hollow cylindrical member 502 has an annular bevel gear 522 on the proximal end thereof in the rotor housing 250 of the coupler 15.

The drive shaft 50b and the driven shaft 53 are supported on the rotor housing 250 by bearings 528, and sealed with respect to the rotor housing 250 by O-rings 530. The hollow cylindrical member 502 is supported on the rotor housing 250 by a slide bearing 532 and sealed with respect to the rotor housing 250 by an O-ring 534. The O-ring 534 is positioned closer to the distal end of the hollow cylindrical member 502 than the slide bearing 532, in order to prevent bending loads produced by the motor 40b from being imposed on the O-ring 534.

The hollow cylindrical member 502 is rotatably supported by the slide bearings 514, 532 respectively at distal and proximal ends thereof, and hence is stably rotatable. The slide bearings 514, 532 provide a simple and inexpensive support for the hollow cylindrical member 502.

The drive shaft 50b has a drive bevel gear 536 on an end thereof in the Y1 direction, and the driven shaft 53 has a driven bevel gear 538 on an end thereof in the Y2 direction. The drive bevel gear 536 and the driven bevel gear 538 are disposed in respective coaxially facing positions.

The hollow cylindrical member 502 extends to a position, which is slightly spaced toward the distal end thereof, from the axial center of the drive shaft 50b with respect to the Z directions. The bevel gear 522 is held in mesh with the drive bevel gear 536 and the driven bevel gear 538 in a 900 spaced relationship thereto, thereby supporting the hollow cylindrical member 502.

Since the hollow cylindrical member 502 is supported by the drive bevel gear 536 and the driven bevel gear 538 in the Y1 and Y2 directions, the hollow cylindrical member 502 is well balanced and does not impose local loads on the slide bearing 532. The surface pressure on contacting regions of the drive bevel gear 536 and the bevel gear 522 can be increased to reliably transmit power from the motor 40b to the hollow cylindrical member 502.

Operations of the medical manipulator 10 thus constructed will be described below.

The operator operates the trigger lever 32 and the composite input unit 34 of the operating unit 14 depending on the surgical technique to be performed on the patient. The controller 27 reads detected stroke signals from the input sensors 39a, 39b, 39c, which are associated with the trigger lever 32 and the composite input unit 34, and determines target angles for the yaw axis, the roll axis, and the gripper axis. The controller 27 then controls angular displacements of the motors 40a, 40b, 40c in order to enable the mechanisms of the distal-end working unit 12 to achieve the target axes.

For opening and closing the gripper 60, the motor 40b is energized to cause the hollow cylindrical member 502, the gear 334, and the gear ring 352 to turn the gear 374.

The gripper 60, or an opening and closing mechanism such as scissors or the like that may be used in place of the gripper 60, is a mechanism for performing actual operations, and often is required to exert relatively large forces. Since the gripper 60 is actuated by the hollow cylindrical member 502, the gripper 60 or others can produce large forces with high power transmission efficiency.

For rotating the distal-end working unit 12 about the roll axis, i.e., about the second rotational axis Or, the motor 40c is energized to actuate the drive base 350, and the motor 40b also is energized to cancel out interfering movements of the gripper 60.

Since the motor 40c is not limited to any rotational range, the motor 40c can rotate the distal-end working unit 12 endlessly within an infinite angular range about the roll axis. Since the hollow cylindrical member 502 also is not limited to any rotational range, the hollow cylindrical member 502 can cancel out interfering movements of the gripper 60 limitlessly, depending on the rotation of the distal-end working unit 12 about the roll axis.

The motor 40c is directly coupled to the drive base 350 through the speed reducer 42c and the fastening pin 356. Inasmuch as substantially no power transmission loss is caused between the motor 40c and the drive base 350, the motor assembly 363 is highly efficient and responsive.

For turning the distal-end working unit 12 about the yaw axis, i.e., the first rotational axis Oy, the motor 40a is energized to cause the proximal-end pulley 50a, the wire 54, and the distal-end pulley 330 to swing the motor holding tube 328. The motors 40b, 40c also are energized to cancel out interfering movement of the gripper 60 and interfering rotation of the distal-end working unit 12 about the roll axis.

For easier understanding of the invention, the operation of the gripper 60 about the gripper axis, rotation of the distal-end working unit 12 about the roll axis, and swinging movements of the distal-end working unit 12 about the yaw axis, have individually been described above. However, the gripper 60 and the distal-end working unit 12 can be operated simultaneously in a combined fashion.

The medical manipulator 10 according to the present invention offers the following advantages. Since the motor 40c is not limited to any rotational range, it can rotate the distal-end working unit 12 endlessly about the roll axis. As the motor 40c is combined with the speed reducer 42c, the motor 40c can produce a high torque even though it is small in size, and can easily perform angular control about the roll axis.

Inasmuch as the motor 40c is positioned closer to the distal end of the distal-end working unit 12 than the yaw axis, the motor 40c can appropriately rotate the distal-end working unit 12 about the roll axis, even when the distal-end working unit 12 is turned about the yaw axis, because the motor 40c itself also is turned about the yaw axis.

The motor holding tube 328 in which the motor 40c is fixedly mounted is coaxial with the roll axis, and is rotatably supported by the shaft 312 coaxial with the yaw axis. The motor 40c is stably held by the motor holding tube 328 and can be angularly moved about the yaw axis.

Since the output shaft 363a of the motor 40c is coaxial with the roll axis, the roll-axis mechanism 302 for rotating the distal-end working unit 12 about the roll axis is simple in structure.

The gear ring 352 is positioned around the motor 40c as a coaxial power transmitting tubular body, with the face gears 370, 372 on axial opposite ends thereof. The distal-end working unit 12 includes the gripper 60, which is positioned closer to the distal end thereof than the yaw axis (Oy, the shaft 312) and the roll axis (Or, the output shaft 363a). The gripper 60 is actuated by the gear ring 352. Therefore, the gripper 60, which is positioned on the distal end of the distal-end working unit 12, can be actuated appropriately.

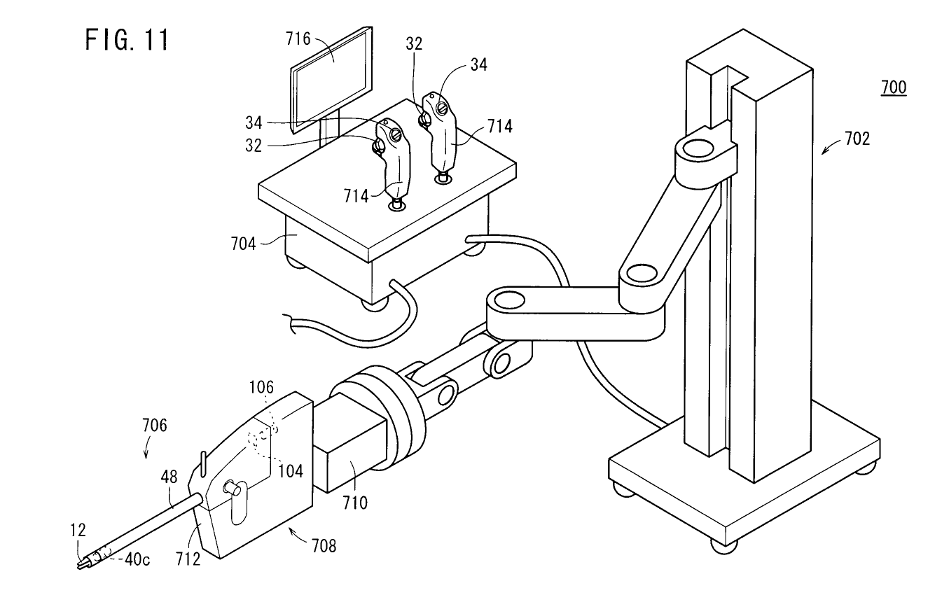

The medical manipulator 10 may be applied to a surgical robot system 700, as shown in FIG. 11, for example.

The surgical robot system 700 has an articulated robot arm 702 and a console 704, with a working unit 706 connected to the distal end of the robot arm 702. The distal end of the robot arm 702 incorporates therein a manipulator 708, which has the same mechanisms as those of the medical manipulator 10. The manipulator 708 comprises the working unit 706. The robot arm 702 makes up a means for moving the working unit 706, and is not limited to an installed type, but may be an autonomous movable type. The console 704 may be a table type, a control panel type, or the like.

The robot arm 702 should preferably have six or more independent joints (rotary shafts, slide shafts, etc.) for setting the position and orientation of the working unit 706 as desired. The manipulator 708 at the distal end is integrally combined with a distal end 710 of the robot arm 702. Instead of the actuator block 30 (see FIG. 1) described above, the manipulator 708 has an actuator block 712, which is connected to the distal end 710 and incorporates the motors 40a, 40b therein.

The robot arm 702 operates under control of the console 704, and may be automatically actuatable according to a program, actuated by joysticks 714 mounted on the console 704, or actuated by a combination of the program and the joysticks 714. The console 704 includes the function of the controller 27 (see FIG. 1). The working unit 706 includes the distal-end working unit 12 described above, which incorporates the motor 40c for rotating the distal-end working unit 12 about the roll axis.

The console 704 includes the two joysticks 714 serving as an operation command unit, and a monitor 716. Although not shown, the two joysticks 714 are capable of individually operating two robot arms 702. The two joysticks 714 are disposed in respective positions where they can easily be operated by both hands of the operator. The monitor 716 displays information such as an image produced by an endoscope.

The joysticks 714 can be moved vertically and horizontally, twisted, and tilted, and the robot arm 702 can be moved depending on such movements of the joysticks 714. The joysticks 714 may be master arms. The robot arm 702 and the console 704 may communicate with each other via a communication means comprising a wired link, a wireless link, a network, or a combination thereof.

Although certain preferred embodiments of the present invention have been shown and described in detail, it should be understood that various changes and modifications may be made to such embodiments without departing from the scope of the invention as set forth in the appended claims.

What is claimed is:

1. A medical manipulator comprising:
an actuator having a plurality of proximal-end motors; and
a working unit detachably mounted on the actuator by a coupler;
the working unit comprising:
a hollow joint shaft extending from the coupler;
a distal-end working unit mounted on a distal end of the hollow joint shaft and having at least three axes including a roll axis about which the distal-end working unit is rotatable; and
a distal-end motor mounted in the distal-end working unit,
wherein the distal-end working unit is operated about the roll axis by the distal-end motor, and is operated about the at least three axes other than the roll axis by the proximal-end motors.

2. A medical manipulator according to claim 1, wherein the at least three axes include a yaw axis, which is positioned closer to a proximal end of the distal-end working unit than the roll axis, and the distal-end motor is positioned closer to a distal end of the distal-end working unit than the yaw axis.

3. A medical manipulator according to claim 2, wherein the distal-end working unit comprises:
a tubular body coaxial with the roll axis and angularly movable about the yaw axis,
the distal-end motor being disposed in the tubular body.

4. A medical manipulator according to claim 1, wherein the distal-end motor has an output shaft coaxial with the roll axis.

5. A medical manipulator according to claim 4, wherein the distal-end working unit comprises:
a power transmitting tubular body coaxially disposed around the distal-end motor and having face gears respectively on axial ends thereof; and
a gripper openable and closable about a gripper axis positioned closer to a distal end of the distal-end working unit than the output shaft,
wherein the gripper is opened and closed about the gripper axis by rotation of the power transmitting tubular body.

6. A medical manipulator according to claim 1, wherein the working unit comprises:
a proximal-end rotor rotatable by the proximal-end motor;
a distal-end rotor disposed in the distal-end working unit for operating the distal-end working unit about one of the at least three axes other than the roll axis; and
a flexible member trained around the proximal-end rotor and the distal-end rotor for transmitting power from the proximal-end rotor to the distal-end rotor.

7. A medical manipulator according to claim 1, further comprising a hollow cylindrical member disposed in the joint shaft and rotatable by one of the distal-end motors for operating the distal-end working unit about one of the at least three axes other than the roll axis.
